(19) United States
(12) Patent Application Publication
(10) Pub. No.: US 2026/0356864 A1
(43) Pub. Date: Nov. 12, 2026

(54) NEW INTERMEDIATE FOR ß-CAROTENE SYNTHESIS

(71) Applicant: DSM IP Assets B.V., Maastricht (NL)

(72) Inventors: Werner BONRATH, Kaiseraugst (CH); Patrick FREY, Kaiseraugst (CH); Leonardo LE, Kaiseraugst (CH); Christian LOTHSCHUETZ, Kaiseraugst (CH); Bettina WUESTENBERG, Kaiseraugst (CH)

(21) Appl. No.: 19/116,023

(22) PCT Filed: Sep. 25, 2023
(86) PCT No.: PCT/EP2023/076344

§ 371 Date: Mar. 27, 2025

(30) Foreign Application Priority Data
Sep. 29, 2022 (EP) 22020466.3

(51) Int. Cl. C07F 9/54; C07C 403/00

(52) U.S. Cl. CPC C07F 9/5442; C07C 403/00; C07C 2601/16

(57) ABSTRACT

The present invention relates to a new intermediate, which can be used in the process for producing β-carotene.

The present invention relates to a new intermediate, which can be used in the process for producing β-carotene.

β-Carotene is an organic, strongly coloured red-orange pigment abundant in fungi, plants, and fruits. β-Carotene is an important product with many different ways of applications.

β-Carotene is the compound of the following formula (I)

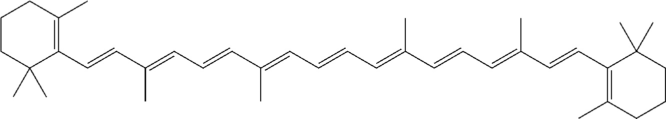 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

β-Carotene is a member of the carotenes, which are terpenoids (isoprenoids), synthesized biochemically from eight isoprene units and thus having 40 carbons. Among the carotenes, β-carotene is distinguished by having beta-rings at both ends of the molecule.

β-Carotene is the most common form of carotene found in plants.

When used as a food colouring, it has the E number E160a (ii).

Furthermore, in nature, β-carotene is a precursor (inactive form) to vitamin A via the action of beta-carotene 15,15′-monooxygenase.

β-Carotene is a compound that gives vivid yellow, orange, and red colouring to vegetables. The human body converts β-carotene into vitamin A (retinol).

Next to its dyeing properties β-carotene has also some health benefits, such as effects on eye health, on improved cognitive function, on skin protection and on cancer prevention.

A common way to produce β-carotene is shown in the following scheme

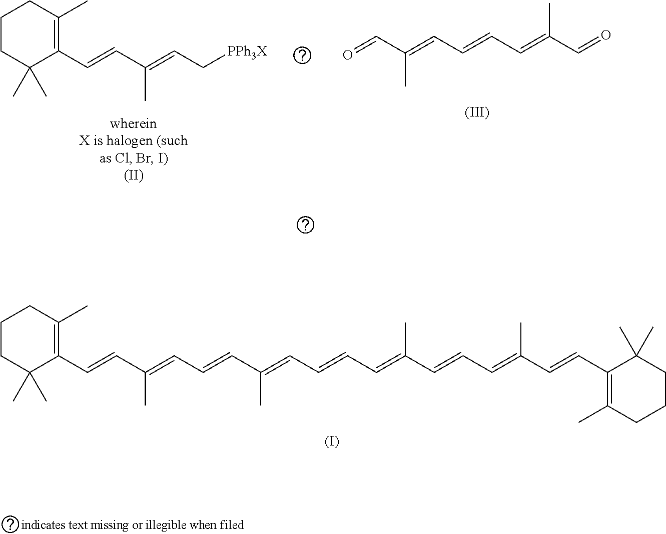

Due to its importance, there is always a need for an improved way to obtain β-carotene.

Surprisingly, we found that the use of a new intermediate, which has no halogen ion, leads to an excellent yield and excellent purity of the resulting β-carotene.

The following vinyl salt of formula (IV)

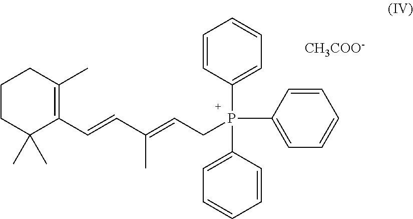

is a new compound, which can be used in the synthesis of β-carotene.

Therefore, the present invention relates to the compound of formula (IV)

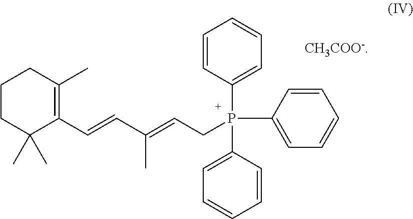

In the context of the present invention, all disclosed compounds (represented by the chemical formulae) can be in any possible stereochemical configuration.

The new compound of formula (IV) can be produced according to the following process:

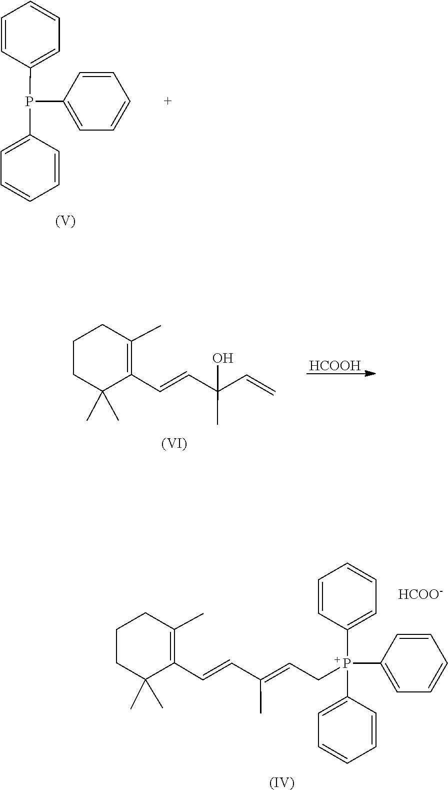

Therefore, the present invention relates to the process (P) of production of the compound of formula (IV)

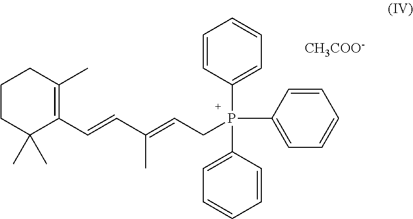

by reacting the compound of formula (V)

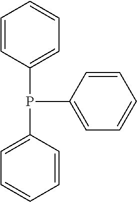 [SMILES: c1ccc(P(c2ccccc2)c2ccccc2)cc1]

with the compound of formula (VI)

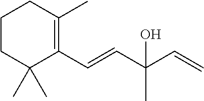 [SMILES: C=CC(C)(O)/C=C/C1=C(C)CCCC1(C)C]

in the presence of acetic acid.

The compound of formula (V) and (VI) are available commercially from a variety of suppliers. Alternatively, they could also be synthesised using suitable starting materials.

The process to produce the compound of formula (IV) is carried out in acetic acid. No additional inert solvent is needed.

Optionally, an inert solvent could be used as well.

Usually and preferably acetic acid (CH₃COOH) is used in molar excess in regard to the compound of formula (V).

Therefore, the present invention relates to the process (P1), which process (P), wherein acetic acid is used in molar excess in view of the compound of formula (V).

Usually the compound of formula (VI) is used in a molar ratio of 1 to 1.5. (in view of the compound of formula (V).

This means that the compound of formula (VI) can be used in an equimolar amount or in a slight excess in view of the compound of formula (V).

Therefore, the present invention relates to the process (P2), which process (P) or (P1), wherein the compound of formula (VI) is used in a molar ratio of 1 to 1.5. (in view of the compound of formula (V).

The process according to the present invention is usually carried out at elevated temperatures.

Usually, the process according to the present invention is carried out at a temperature range of from 30 to 120 °C.

Therefore, the present invention relates to the process (P3), which process (P), (P1) or (P2), wherein the process is carried out at a temperature range of from 30 to 120 °C.

The new compound of formula (IV) is used in a process to produce β-carotene.

The following reaction scheme shows how to obtain β-carotene when using the compound of formula (IV) as starting material:

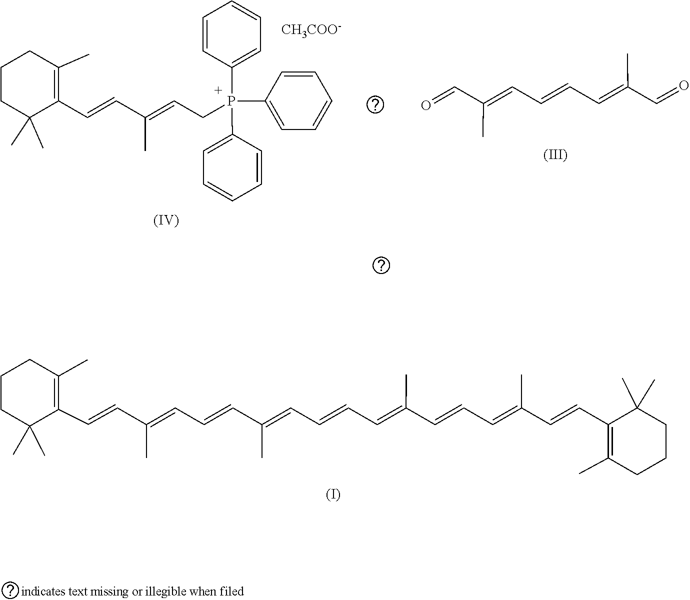

Therefore, the present invention relates to the use of the compound of formula (IV) to produce β-carotene.

Therefore, the present invention relates to a reaction process (RP) to produce β-carotene (compound of formula (I))

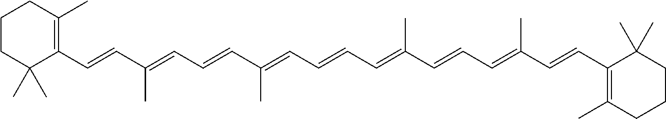 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

by reacting the compound of formula (IV)

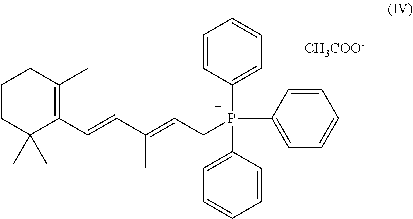

with the compound of formula (III)

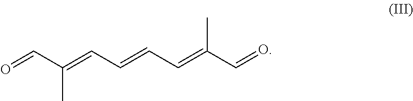

This reaction can be carried using the known reaction conditions as disclosed for the compound of formula (II) as starting material.

The compound of formula (IV) is used in a molar ratio of at least 2:1 (in regard to the compound of formula (III)).

Therefore, the present invention relates to the process (RP1), which is the process (RC), wherein the compound of formula (IV) is used in a molar ratio of at least 2:1 (in regard to the compound of formula (III)).

The process to produce β-carotene is usually carried out at a temperature of between 0-150 °C. Preferably, the process is carried out at a range of 5 °C to 130 °C.

Therefore, the present invention relates to the process (RP2), which is the process (RC) or (RP1), wherein the process is carried out at a temperature of between 0-150 °C.

Therefore, the present invention relates to the process (RP2′), which is the process (RC) or (RP1), wherein the process is carried out at a temperature of between 5 °C to 130 °C.

A base can be added as well to the reaction mixture.

The base is usually an alkali hydroxide, earth alkali hydroxide, alkali carbonate, earth alkali carbonate, KF/Al₂O₃, NaOCH₃ and KOCH₃.

Preferably, the base is CsOH, KOH, NaOH, Na₂CO₃ or K₂CO₃.

The base is usually used in molar excess in view of the compound of formula (III). Usually 2-20 mol-equivalent in view of the compound of formula (III).

Therefore, the present invention relates to a process (RP3), which is process (RP), (RP1), (RP2) or (RP2′), wherein the process is carried out in the presence of at least one base.

Therefore, the present invention relates to a process (RP3′), which is process (RP3), wherein the base is chosen from the group consisting of alkali hydroxide, earth alkali hydroxide, alkali carbonate, earth alkali carbonate, KF/Al₂O₃, NaOCH₃ and KOCH₃.

Therefore, the present invention relates to a process (RP3″), which is process (RP3), wherein the base is chosen from the group consisting of CsOH, KOH, NaOH, Na₂CO₃ and K₂CO₃.

The process to produce β-carotene is usually and preferably carried out in a solvent.

Therefore, the present invention relates to a process (RP4), which is process (RP), (RP1), (RP2), (RP2′), (RP3), (RP3′) or (RP3″), wherein the process is carried out in the in at least one solvent.

Such a solvent can be any commonly known solvents used in the processes disclosed and described in the prior art.

Suitable solvents are CH₂Cl₂, CHCl₃, linear or branched C₁-C₄ alcohols, toluene, mixtures of alcohols/C₅-C₇ alkanes/water.

Preferred solvents are CH₂Cl₂ or CH₃CH₂OH.

Therefore, the present invention relates to a process (RP5), which is process (RP4), wherein the solvent is chosen from the group consisting of . . . .

Very preferred solvents are alcohols represented by the compound of formula (VII)

R—OH (VII),
wherein
R is a linear or branched C₃-C₁₀-alkyl moiety (preferably C₄-C₈-alkyl, more preferred C₄-C₇-alkyl, most preferred C₅alkyl).

Most preferably, 1-pentanol, 2-pentanol, 2-methyl-butan-1-ol, 2-methyl-2-butanol and/or 3-methyl-1-butanol is used as solvent.

Therefore, the present invention relates to a process (RP6), which is process (RP4), wherein the solvent is at least one compound of formula (VII)

R—OH (VII),
wherein
R is a linear or branched C₃-C₁₀-alkyl moiety (preferably C₄-C₈-alkyl, more preferred C₄-C₇-alkyl, most preferred C₅alkyl).

Therefore, the present invention relates to a process (RP6′), which is process (RP6), wherein the alcohol of the compound of formula (VII) is chosen from the group consisting of 1-pentanol, 2-pentanol, 2-methyl-butan-1-ol, 2-methyl-2-butanol and 3-methyl-1-butanol.

Another group of suitable solvents are organic carbonates.

Such organic carbonates have the following formula (VIII)

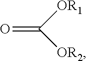

wherein
R₁ is a C₁-C₄alkyl moiety, and R₂ is a C₁-C₄alkyl moiety.

Preferably, organic carbonates of compound of formula (VIII) are those, wherein
R₁ is a C₁-C₂alkyl moiety, and
R₂ is a C₁-C₂alkyl moiety,
   as solvent.

More preferably, organic carbonates of compound of formula (VIII) are chosen from the group consisting of dimethylcarbonate and diethylcarbonate as solvent.

Most preferably, the organic carbonate of formula (VIII) is diethylcarbonate.

Therefore, the present invention relates to a process (RP7), which is process (RP4), wherein the solvent is at least one organic carbonate.

Therefore, the present invention relates to a process (RP7′), which is process (RP7), wherein the organic carbonate is a compound of formula (VIII)

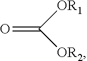

wherein
R₁ is a C₁-C₄alkyl moiety, and
R₂ is a C₁-C₄alkyl moiety.

Therefore, the present invention relates to a process (RP7′), which is process (RP7), wherein the organic carbonates are chosen from the group consisting of dimethylcarbonate and diethylcarbonate.

Therefore, the present invention relates to a process (RP7′), which is process (RP7), wherein the organic carbonate is diethylcarbonate.

The following Examples illustrate the invention further without limiting it. All percentages and parts, which are given, are related to the weight and the temperatures are given in °C, and the pressures are absolute pressures when not otherwise stated.

### EXAMPLES

### Example 1: Synthesis Compound of Formula (IV)

At room temperature and under argon atmosphere, triphenylphosphine (103.9 g, 1.1 eq., 392.2 mmol) was suspended in acetic acid (183.7 mL, 9 eq., 3.209 mol) and heated to 60 °C in an oil-bath. At this temperature, vinylol (83 mL, 1.00 eq., 356.5 mmol) was added dropwise within 1 hour via syringe pump. After complete addition, stirring was continued for another 2 hours. Then, the orange solution was cooled within 30 minutes to room temperature, transferred to a separation funnel and diluted with methanol (300 mL). Hexane (300 mL) was added. The mixture was shaken, and the layers were separated. The methanol layer was extracted with hexane (300 mL) and the hexane layer was washed with methanol (100 mL). The methanol layers were combined and evaporated to dryness under reduced pressure. The product was obtained as an orange oil (278.0 g, 56.2%) in 83.5% yield. E/Z=9:1.

### Example 2: Synthesis of β-Carotene

At room temperature and under argon atmosphere, a solution of vinyl salt acetate (8.71 g, 2.1 eq., 10.7 mmol), obtained from Example 1, in 1-pentanol (20 mL) was adjusted to pH 7 by addition of NaOH 5% (3 mL) and NaOH 10% (10.6 mL). To this solution was added C10-dialdehyde (841 mg, 1 eq., 5.10 mmol), which is the compound of formula (III), and 1-pentanol (5 mL). The yellow suspension was cooled to 0 °C (ice-bath), and NaOH (4.5 g, 4.1 mL, 10 wt %,) was added. Then the cooling bath was removed and replaced with an oil-bath. The reaction mixture was warmed to 40 °C and stirred for 1 hour and then for another 1 hour at reflux (100 °C). The oil-bath was removed, and the reaction mixture was cooled to 40 °C within 30 min. Distilled water (8 ml) was added over 10 min and stirring was continued for 16 hours. After that, the red suspension was cooled to 0 °C, stirred for 30 min and filtered. The filter cake was rinsed with 1-pentanol (8 mL) and distilled water (8 mL). The red crystals were dried under vacuum at 50 °C. Dark purple crystals (2.45 g, 91.2 wt %) were obtained in 81.6% yield.

### Example 3: Synthesis of β-Carotene

At room temperature and under argon atmosphere, vinyl salt acetate (5.00 g, 2.1 eq., 5.88 mmol), obtained from Example 1, and C10-dialdehyde (462 mg, 1 eq., 2.80 mmol), which is the compound of formula (III), were dissolved in isoamyl alcohol (20 mL). The yellow suspension was cooled to 0 °C (ice-bath), and NaOH (15%) (9.0 g, 7.7 mL, 12 eq., 33.6 mmol) was added dropwise over 4 hours. Then the cooling bath was removed and replaced with an oil-bath. The reaction mixture was warmed to 40 °C and stirred for 1 hour followed by another 20 hours at reflux (99 °C). The oil-bath was removed, and the reaction mixture was cooled to 40 °C within 30 min. Distilled water (10 ml) was added over 10 min. After that, the red suspension was cooled to 0 °C, stirred for 30 min and filtered. The filter cake was rinsed with distilled water (3×10 mL). The red crystals were dried under vacuum at 50 °C. Red crystals were obtained (1.20 g, 90.6 wt %) in 72.3% yield.

### Example 4: Synthesis of β-Carotene

At room temperature and under argon atmosphere, C10-dialdehyde (457 mg, 1 eq., 2.77 mmol), which is the compound of formula (III), and potassium carbonate (4.64 g, 12 eq., 33.3 mmol) were suspended in isoamyl alcohol (7 mL). The yellow suspension was warmed to 40 °C (oil-bath), and a freshly prepared solution of vinyl salt acetate (5.00 g, 2.1 eq., 5.82 mmol), obtained from Example 1, in isoamyl alcohol (10 mL) was added dropwise over 20 min. Then the reaction mixture was heated to 80 °C and stirred for 4 hours. Distilled water (15 mL) was added with an addition funnel. After that, the reaction mixture was heated to 100 °C and stirred for 1 hour. The oil-bath was removed, and the reaction mixture was cooled to 40 °C within 30 min. Distilled water (10 ml) was added over 10 min. After that, the red suspension was cooled to 0 °C, stirred for 30 min and filtered. The filter cake was rinsed with isoamyl alcohol (5 mL) and distilled water (3×10 mL). The dark red crystals were dried under vacuum at 50 °C. Red crystals were obtained (1.05 g, 92.7 wt %) in 65.4% yield.

### Example 5: Synthesis of β-Carotene

At room temperature and under argon atmosphere, C10-dialdehyde (444 mg, 1 eq., 2.70 mmol), which is the compound of formula (III), and potassium carbonate (4.52 g, 12 eq., 32.3 mmol) were suspended in methanol (7 mL). The yellow suspension was cooled to 0 °C (ice-bath), and a freshly prepared solution of vinyl salt acetate (5.00 g, 2.1 eq., 5.66 mmol), obtained from Example 1, in methanol (10 mL) was added dropwise over 20 min. Then the reaction mixture was heated to 65 °C (oil-bath) and stirred for 4 hours. The oil-bath was removed, and the reaction mixture was cooled to 40 °C within 30 min. Distilled water (25 mL) was added with an addition funnel (20 min). After that, the red suspension was cooled to 0 °C, stirred for 30 min and filtered. The filter cake was rinsed with distilled water (3×10 mL). The dark red crystals were dried under vacuum at 50 °C. Dark red crystals were obtained (3.70 g, 35.6 wt %) in 91.0% yield.

### Example 6: Synthesis of β-Carotene

At room temperature and under argon atmosphere, C10-dialdehyde (462 mg, 1 eq., 2.80 mmol), which is the compound of formula (III), and potassium carbonate (4.69 g, 12 eq., 33.6 mmol) were suspended in 1-propanol (7 mL). The yellow suspension was cooled to 0 °C (ice-bath), and a freshly prepared solution of vinyl salt acetate (5.00 g, 2.1 eq., 5.88 mmol), obtained from Example 1, in methanol (10 mL) was added dropwise over 20 min. Then the reaction mixture was heated to 60 °C (oil-bath) and stirred for 4 hours. After that, reaction mixture was heated to reflux (97 °C) for another hour. The oil-bath was removed, and the reaction mixture was cooled to 40 °C within 30 min. Distilled water (25 mL) was added with an addition funnel (20 min). After that, the red suspension was cooled to 0 °C, stirred for 30 min and filtered. The filter cake was rinsed with distilled water (3×10 mL). The dark red crystals were dried under vacuum at 50 °C. Dark red crystals were obtained (1.70 g, 71.1 wt %) in 80.3% yield.

1. Compound of formula (IV)

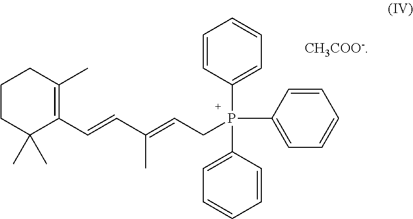

2. Process to produce the compound of formula (I) 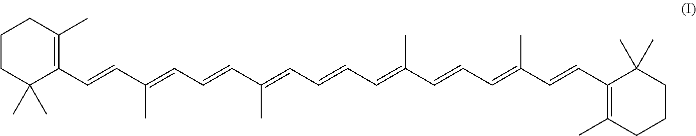 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]
by reacting the compound of formula (IV) 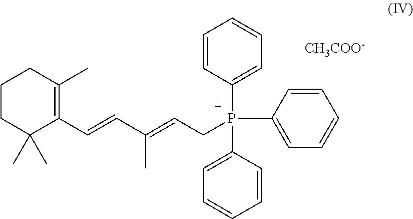
with the compound of formula (III)

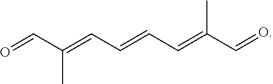

3. Process according to claim 2, wherein the compound of formula (IV) is used in a molar ratio of at least 2:1 (in regard to the compound of formula (III)).

4. Process according to claim 2, wherein the process is carried out at a temperature of between 0-150 °C.

5. Process according to claim 2, wherein the process is carried out in the presence of at least one base.

6. Process according to claim 5, wherein the base is chosen from the group consisting of alkali hydroxide, earth alkali hydroxide, alkali carbonate, earth alkali carbonate, KF/Al₂O₃, NaOCH₃ and KOCH₃.

7. Process according to claim 2, wherein the process is carried out in at least one solvent.

8. Process according to claim 7, wherein the at least one solvent is chosen from the group consisting of CH₂Cl₂, CHCl₃, linear or branched C₁-C₄ alcohols, toluene, mixtures of alcohols/C₅-C₇ alkanes/water.

9. Process according to claim 7, wherein the solvent is at least one compound of formula (VII)
R—OH (VII),
wherein
R is a linear or branched C₃-C₁₀-alkyl moiety.

10. Process according to claim 9, wherein the alcohol is chosen from the group consisting of 1-pentanol, 2-pentanol, 2-methyl-butan-1-ol, 2-methyl-2-butanol and 3-methyl-1-butanol.

11. Process according to claim 7, wherein the at least one solvent is an organic carbonate.

12. Process according to claim 11, wherein the at least one solvent is an organic carbonate of compound of formula (VIII) 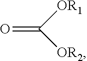
wherein
R₁ is a C₁-C₄alkyl moiety, and
R₂ is a C₁-C₄alkyl moiety.

13. Process according to claim 11, wherein the organic carbonates are chosen from the group consisting of dimethylcarbonate and diethylcarbonate.

14. Process according to claim 11, wherein the organic carbonate is diethylcarbonate.
